SEQUENCE LISTING (22 sequences)

SEQ ID NO: 1 - DNA, 48 bp, Artificial Sequence
  Description of Artificial Sequence adaptor oligonucleotide sequence
tcgaccatggcgcagcagcctggaaaagttcttggggaccaaagaagg

SEQ ID NO: 2 - DNA, 44 bp, Artificial Sequence
  Description of Artificial Sequence adaptor oligonucleotide sequence
ccttctttggtccccaagaacttttccaggctgctgcgccatgg

SEQ ID NO: 3 - DNA, 29 bp, Artificial Sequence
  Description of Artificial Sequence oligonucleotide used for cloning
aattaggatcctcatgggtaccttgtatc

SEQ ID NO: 4 - DNA, 21 bp, Artificial Sequence
  Description of Artificial Sequence oligonucleotide used for cloning
gagccttttgtgatctgtcat

SEQ ID NO: 5 - DNA, 21 bp, Artificial Sequence
  Description of Artificial Sequence oligonucleotide used for cloning
atgacagatcacaaaaggctc

SEQ ID NO: 6 - DNA, 30 bp, Artificial Sequence
  Description of Artificial Sequence oligonucleotide used for cloning
tggggatccgaaaaacataatctggaaagc

SEQ ID NO: 7 - DNA, 42 bp, Artificial Sequence
  Description of Artificial Sequence oligonucleotide used for cloning
aaggatccgccaccatggattctctaccagatgaattttttg

SEQ ID NO: 8 - DNA, 24 bp, Artificial Sequence
  Description of Artificial Sequence oligonucleotide used for cloning
caatacccagtgaatcttcaacag

SEQ ID NO: 9 - DNA, 32 bp, Artificial Sequence
  Description of Artificial Sequence oligonucleotide used for cloning
attatgtttacgagcaagcctttccttttcag

SEQ ID NO: 10 - DNA, 33 bp, Artificial Sequence
  Description of Artificial Sequence oligonucleotide used for cloning
cttgctcgtaaacataatctggaaagcacaatg

SEQ ID NO: 11 - DNA, 31 bp, Artificial Sequence
  Description of Artificial Sequence oligonucleotide used for cloning
aattaggatccatggaagacaagttctatag

SEQ ID NO: 12 - DNA, 33 bp, Artificial Sequence
  Description of Artificial Sequence oligonucleotide used for cloning
gcgaattcttacttgactttgtttatttgttca

SEQ ID NO: 13 - DNA, 30 bp, Artificial Sequence
  Description of Artificial Sequence oligonucleotide used for cloning
caaaggatcccaaggatttgccttgagtac

SEQ ID NO: 14 - DNA, 31 bp, Artificial Sequence
  Description of Artificial Sequence oligonucleotide used for cloning
gggaattcttatccttcccatgtggcaggac

SEQ ID NO: 15 - DNA, 29 bp, Artificial Sequence
  Description of Artificial Sequence oligonucleotide used for cloning
tggggatccgccagtaatccagatgagga

SEQ ID NO: 16 - DNA, 26 bp, Artificial Sequence
  Description of Artificial Sequence oligonucleotide used for cloning
atgaattcatgggtaccttgtatcag

SEQ ID NO: 17 - DNA, 32 bp, Artificial Sequence
  Description of Artificial Sequence oligonucleotide used for cloning
tcgaattcttattttccataagcatccttcaa

SEQ ID NO: 18 - DNA, 30 bp, Artificial Sequence
  Description of Artificial Sequence oligonucleotide used for cloning
actggatcctggcaagaaaaccaaatgcag

SEQ ID NO: 19 - DNA, 32 bp, Artificial Sequence
  Description of Artificial Sequence oligonucleotide used for cloning
aggaattcttataagaactgctgcatcagtat

SEQ ID NO: 20 - DNA, 31 bp, Artificial Sequence
  Description of Artificial Sequence oligonucleotide used for cloning
atgaattcttaattcatgatggagggatcaa

SEQ ID NO: 21 - protein, 1213 aa, Homo sapiens
MDSLPDEFFVRHPAVEDQRKEETENKLEKSSGQLNKQENDIPTDLVPVNLLLEVKKLLNAINTLPKGVVPHIKKFLQEDFSFQTMQREVAANSQNGEEIVPALTLRFLITQLEAALRNIQAGNYTAHQINIGYYLTLLFLYGVALTERGKKEDYTEAENKFLVMKMMIQENEICENFMSLVYFGRGLLRCAQKRYNGGLLEFHKSLQEIGDKNDHWFDIDPTEDEDLPTTFKDLLNNFIKTTESNIMKQTICSYLDCERSCEADILKNTSYKGFFQLMCSKSCCVYFHKICWKKFKNLKYPGENDQSFSGKKCLKEGCTGDMVRMLQCDVPGIVKILFEVVRKDEYITIENLGASYRKLISLKITDTDIRPKISLKFNTKDEMPIFKLDYNYFYHLLHIIIISGTDIVRQIFDEAMPPPLLKKELLIHKNVLESYYNHLWTNHPLGGSWHLLYPPNKELPQSKQFDLCLLLALIKHLNVFPAPKKGWNMEPPSSDISKSADILRLCKYRDILLSEILMNGLTESQFNSIWKKVSDILLRLGMMQEDIDKVKENPIENISLDYHQLSVYLGIPVPEIIQRMLSCYQQGIALQSITGSQRIEIEELQNEEEELSPPLMEYNINVKSHPEIQFAKINKDGTSIPSESSTESLKDLQEVKSKQRKKKKTKNKKNKDSKEDQVPYVVEKEEQLRKEQANPHSVSRLIKDDASDVQEDSAMEDKFYSLDELHILDMIEQGSAGKVTTDYGETEKERLARQRQLYKLHYQCEDFKRQLRTVTFRWQENQMQIKKKDKIIASLNQQVAFGINKVSKLQRQIHAKDNEIKNLKEQLSMKRSQWEMEKHNLESTMKTYVSKLNAETSRALTAEVYFLQCRRDFGLLHLEQTEKECLNQLARVTHMAASNLESLQLKAAVDSWNAIVADVRNKIAFLRTQYNEQIMKVKQGFALSTLPPVQLPPPPPSPEILMQQFLGRPLVKESFFRPILTVPQMPAVCPGVVSATGQPRAPLMTGIAWALPAPVGDAVPPSAGLRIDPSIMNWERITDRLKTAFPQQTRKELTDFLRKLKDAYGKSLSELTFDEIVCKISQFIDPKKSQSQGKSVSNVNCVSPSHSPSQPDAAQPPKPAWRPLTSQGPATWEGASNPDEEEEEEEPCVICHENLSPENLSVLPCAHKFHAQCIRPWLMQQGTCPTCRLHVLLPEEFPGHPSRQLPRSDTRYP

SEQ ID NO: 22 - protein, 499 aa, Homo sapiens
MEDKFYSLDELHILDMIEQGSAGKVTTDYGETEKERLARQRQLYKLHYQCEDFKRQLRTVTFRWQENQMQIKKKDKIIASLNQQVAFGINKVSKLQRQIHAKDNEIKNLKEQLSMKRSQWEMEKHNLESTMKTYVSKLNAETSRALTAEVYFLQCRRDFGLLHLEQTEKECLNQLARVTHMAASNLESLQLKAAVDSWNAIVADVRNKIAFLRTQYNEQINKVKQGFALSTLPPVQLPPPPPSPEILMQQFLGRPLVKESFFRPILTVPQMPAVCPGVVSATGQPRAPLMTGIAWALPAPVGDAVPPSAGLRIDPSIMNWERITDRLKTAFPQQTRKELTDFLRKLKDAYGKSLSELTFDEIVCKISQFIDPKKSQSQGKSVSNVNCVSPSHSPSQPDAAQPPKPAWRPLTSQGPATWEGASNPDEEEEEEEPCVICHENLSPEMLSVLPCAHKFHAQCIRPWLMQQGTCPTCRLHVLLPEEFPGHPSRQLPRSDTRYP

(19) United States
(12) Patent Application Publication
(10) Pub. No.: US 2004/0092441 A1
(43) Pub. Date: May 13, 2004

(54) Tyrosine kinase modulators

(76) Inventors: Giulio Superti-Furga, Heidelberg-Ziegelhausen (DE); Marina Moro, Uppsala (SE); Daniela Barila, Roma (IT); Wilhelmina Pluk, Cuijk (NL)

Correspondence Address: WENDEROTH, LIND & PONACK, L.L.P., 2033 K STREET N. W., SUITE 800, WASHINGTON, DC, 20006-1021, US

(21) Appl. No.: 10/297,382

(22) PCT Filed: Jun. 6, 2001
(86) PCT No.: PCT/IB01/01165

(30) Foreign Application Priority Data
Jun. 6, 2000 (GB) 0013807.3

(51) Int. Cl. A61K 38/17

(52) U.S. Cl. 514/12

(57) ABSTRACT

The invention relates to novel proteins that modulate the activity of tyrosine kinases. The invention also relates to the use of tyrosine kinase modulator proteins in the treatment and diagnosis of cancer in mammals, including humans.

The present invention relates to novel proteins that modulate the activity of tyrosine kinases. The invention also relates to the use of tyrosine kinase modulator proteins in the treatment and diagnosis of cancer in mammals, including humans.

All documents mentioned in the text and listed at the end of the description are incorporated herein by reference.

Protein tyrosine kinases are enzymes that transfer the terminal phosphate of adenosine triphosphate (ATP) to a specific tyrosine residue on a target protein. These enzymes are found in all multicellular organisms and play a central role in the regulation of cellular growth and in the differentiation of complex eukaryotes.

There are two major classes of tyrosine kinases: transmembrane receptor tyrosine kinases and non-receptor tyrosine kinases. Regulation of all protein tyrosine kinases is essential for normal cellular differentiation and proliferation. While controlled activation of tyrosine kinases promotes normal proliferation, deregulated tyrosine kinases can cause neoplastic transformation. Examples from both classes of kinases have been shown to function as dominant oncogenes, generally as a result of overexpression and/or structural alteration.

Transmembrane receptor tyrosine kinases are activated directly by binding of peptide growth factors and cytokines to their extracellular domains. Tyrosine kinases which fall within this class include receptors for platelet-derived growth factor, fibroblast growth factors, hepatocyte growth factor, insulin, insulin-like growth factor-1, nerve growth factor, vascular endothelial growth factor and macrophage colony stimulating factor. The; normal function of these receptors is to act as transducers of extracellular signals.

Some non-receptor tyrosine kinases are associated with cell surface receptors which do not have intrinsic tyrosine kinase activity. For example, members of the Src family of non-receptor protein tyrosine kinases in mammals (such as src, yes, fgr, fyn, ick lyn, hck and blk) are all located on the cytoplasmic side of the plasma membrane, held there partly by their interaction with transmembrane receptors and partly by covalently-attached lipid chains. These proteins are also involved in signal transduction pathways. However, not all non-receptor protein tyrosine kinases are associated with transmembrane receptors. Some are found in the cytoplasm or even in the nucleus of cells. The role of these proteins is in many cases unknown.

The c-Abl protein tyrosine kinase is another example of a non-receptor protein tyrosine kinase. It was originally isolated as a cellular homologue of the v-abl oncogene of a transforming retrovirus, the Abelson murine leukaemia virus. Cellular Abl sequences have now been isolated in humans, D. melanogaster and C. elegans and this gene is now known to be expressed ubiquitously in vertebrates. A further Abl-related gene, arg, has also been isolated from the human genome.

The 60 kDa N-terminal domain of the c-Abl protein is homologous to Src and other Src family members. The sequence includes a myristoylation signal, an SH3 domain, an SH2 domain and a catalytic domain. The large C-terminal region, which is approximately 90 kDa in size is unique to c-Abl and includes a DNA binding domain, a nuclear localisation signal, an actin domain and several proline-rich interaction sites for SH3 domain-containing molecules. This C-terminal domain is fairly divergent from the C-terminal domain of Arg.

Although Abl was first identified 20 years ago, its role is still unclear. Studies of the subcellular location of Abl and mutational analysis have led to it being attributed potential roles in a wide range of cellular processes, including cell-cycle regulation, stress responses, integrin signalling and neural development (see van Etten et al, 1999 for review). The protein is in the main located in the nucleus but a significant fraction is located in the cytoplasm where it associates with filamentous actin and the plasma membrane.

The wild-type c-Abl protein does not transform fibroblasts when overexpressed, suggesting its kinase activity is tightly regulated. However, alterations in the c-abl gene can activate its oncogenic potential. In man, chromosomal translocations between the breakpoint cluster region (BCR) and abl are associated with chronic myelogenous leukaemias and some acute lymphocytic leukaemias (see Sawyers, 1992 for review). Deletion of the SH3 domain also renders c-Abl oncogenic.

The normal mechanism by which c-Abl is regulated in vivo is also still unclear. It has been suggested that the SH3 domain of Abl exerts a negative effect on kinase activity. Given that it has been shown that c-Abl and SH3-mutated Abl have identical tyrosine kinase activity in vitro, it has been suggested that further proteins might be involved in this regulation in vivo (Mayer & Baltimore, 1993).

For example, it has been proposed that an inhibitor might function to stabilise binding between the Abl SH3 domain and linker proline sites. Several proteins known to bind the SH3 domain of Abl, such as Abi-1, Abi-2 and Aap-2 have been proposed as potential inhibitors (van Etten, 1999). However, these proteins appear to act as effectors of c-Abl rather than inhibitors which therefore lessens their therapeutic potential.

Alternative experiments support the view that no cellular inhibitor is required to maintain normal regulation of Abl in vivo. Work carried out by the inventors demonstrated that intramolecular interactions between the Abl SH3, SH2 and linker domains contribute to the regulation of Abl in vivo (Barila & Superti-Furga, 1998). Furthermore, recent experiments show that purified c-Abl can be regulated in vitro, suggesting that no cellular inhibitor is required to maintain Abl's regulation in vivo.

Protein tyrosine kinases such as Abl play an essential role in the regulation of normal cellular proliferation and differentiation in multicellular organisms, as evidenced by the common incidence of mutations in genes encoding tyrosine kinase proteins in certain cancers. However, at present, no effective modulator of this family of tyrosine kinases has been identified. Given the importance of tyrosine kinase proteins in mammalian diseases and particularly in cancer, there is an urgent need for effective modulators of protein tyrosine kinase activity.

### SUMMARY OF THE INVENTION

Accordingly, the present invention provides a tyrosine kinase modulator comprising the amino acid sequence given in FIG. 1, a variant thereof or a functional equivalent thereof. The present invention also provides a tyrosine kinase modulator consisting of the amino acid sequence given in FIG. 1. This protein is referred to herein as FABLE (Finger-containing Abl enhancer). The full length protein has a calculated molecular weight of about 139.3 kDa.

This protein contains an N-terminal Zn-finger-like structure, a central coiled-coil domain with similarities to cytoskeletal proteins, a proline-rich domain and a C-terminal RING-finger. This protein appears to be expressed ubiquitously in human tissues and localises mainly but not exclusively to the cytoplasm.

The FABLE protein is thought to modulate certain'tyrosine kinases by binding to the SH3 domain of the tyrosine kinase. Examples of tyrosine kinases that are thought to be modulated by the proteins of the above-described aspects of the invention include Abl, Src and Fyn.

The FABLE protein is thought to bind to Abi at both the catalytic domain and the SH3 domain. The full length FABLE protein has been overexpressed in mammalian cells and found to enhance the overall activity of Abl. When activated forms of Abl (such as ΔSH3-Abl) are co-expressed with FABLE in mammalian cells, Abl becomes hyperactivated and phosphorylates a protein of around 72 kD in size. This protein is not normally detected as being tyrosine-phosphorylated in cells that contain active forms of Abl, suggesting that FABLE modulates the ability of Abl to interact with cellular proteins.

Expression of FABLE is shown herein to induce the activation of c-Abl. Binding of FABLE to Abl appears to be required for this activation, since a FABLE mutant lacking its interaction domain is not capable of activating c-Abl.

These observations suggest that FABLE is a novel anchoring and modulating protein. This protein is thought to be a critical partner of normal cellular and oncogenic versions of Abl tyrosine kinases.

According to a further aspect of the invention there is provided a tyrosine kinase modulator comprising the amino acid sequence given in FIG. 2, a variant thereof or a functional equivalent thereof. The invention also provides a tyrosine kinase modulator protein consisting of the amino acid sequence given in FIG. 2, which is referred to herein as SIA (Sequence Inhibiting Abl). This protein has a calculated molecular weight of about 56.8 kDa and is an N-terminal truncated version of FABLE.

The SIA protein was initially identified in a yeast screen for human proteins capable of counteracting the lethal effect of c-Abl expression in S. pombe. In yeast, therefore, this protein acts as an inhibitor of Abl.

The invention further provides multimeric complexes of the tyrosine kinase modulators of both the above-described aspects of the invention, both as homodimers and as heterodimers, complexed with other proteins.

The tyrosine kinase modulators of the invention are predicted to be useful in the diagnosis and treatment of diseases that are caused by tyrosine kinases. For example, the Abl protein is known to be involved in certain leukaemias. Targeting of the tyrosine kinase modulators described herein may be an effective way to inhibit the effect of oncogenic versions of the tyrosine kinase such as Abl.

Modulating the activity of such proteins may also affect the radio- and chemosensitivity of cells. For example, c-Abl activity may play a role in increasing radiation protection of cells. Compounds such as FABLE, or functional equivalents thereof, may be useful to activate cellular c-Abl and increase radioprotection, for example to protect normal cells during radiotherapy of cancer cells.

The tyrosine kinase modulators of the invention may also be used as diagnostic aids, for example, allowing the detection of aberrant levels or activities of tyrosine kinases such as Abl. Patients showing such abnormal levels would be potential candidates for preventative treatment or for frequent testing for disease states.

As the skilled reader will be aware, variants and functional equivalents of the FABLE and SIA proteins are likely to share the properties of these proteins and these variants and functional equivalents are included within the scope of the present invention. For example, variants of these proteins may include sequences containing amino acid substitutions, insertions or deletions from the sequences explicitly recited herein. Variants with improved function may also be designed through the systematic or directed mutation of specific residues in the protein sequence. One such functional improvement that may be desired will include features such as greater specificity or affinity for the tyrosine kinase target. The term “variant” is also intended to include fragments of the proteins whose sequences are explicitly recited herein in FIGS. 1 and 2.

The term “functional equivalent” is used herein to describe homologous tyrosine kinase modulator proteins or molecules that belong to the same family as the tyrosine kinase modulators identified herein and that retain the ability to modulate tyrosine kinase activity. Two polypeptides are said to be “homologous” if the sequence of one of the polypeptides has a significant degree of identity or similarity to the sequence of the other polypeptide. “Identity” indicates that at any particular position in the aligned sequences, the amino acid residue is identical between the sequences. “Similarity” indicates that, at any particular position in the aligned sequences, the amino acid residue is of a similar type between the sequences. Degrees of identity and similarity can be readily calculated according to methods known in the art (see, for example, Computational Molecular Biology, Lesk, A. M., ed., Oxford University Press, New York, 1988; Biocomputing. Informatics and Genome Projects, Smith, D. W., ed., Academic Press, New York, 1993).

Typically, greater than 20% identity between two polypeptides over the whole sequence of the tyrosine kinase modulator is considered to be an indication of functional equivalence, more preferably, 30%, more preferably 40%, most preferably 50% or more, provided that either the biological activity of the polypeptide as a tyrosine kinase modulator is retained. Preferably, a functionally equivalent polypeptide according to this aspect of the invention exhibits a degree of sequence identity with a polypeptide sequence explicitly identified herein, or with a fragment thereof, of greater than 60%. More preferred polypeptides have degrees of identity of greater than 70%, 80%, 90%, 95%, 98% or 99%, respectively.

Functionally-equivalent polypeptides according to the invention include natural biological variants (for example, allelic variants or geographical variations within the species from which the polypeptides are derived) and mutants (such as mutants containing amino acid substitutions, insertions or deletions) of the polypeptides whose sequences are explicitly recited herein. Such mutants may include polypeptides in which one or more of the amino acid residues are substituted with a conserved or non-conserved amino acid residue (preferably a conserved amino acid residue) and such substituted amino acid residue may or may not be one encoded by the genetic code. Typical such substitutions are among Ala, Val, Leu and Ile; among Ser and Thr, among the acidic residues Asp and Glu; among Asn and Gln; among the basic residues Lys and Arg; or among the aromatic residues Phe and Tyr.

Particularly preferred are variants in which several, i.e. between 5 and 10, 1 and 5, 1 and 3, 1 and 2 or just 1 amino acids are substituted, deleted or added in any combination. Especially preferred are silent substitutions, additions and deletions, which do not alter the properties and activities of the protein. Also especially preferred in this regard are conservative substitutions. “Mutant” polypeptides also include polypeptides in which one or more of the amino acid residues include a substituent group.

One example of a known protein that bears sufficient sequence homology to the FABLE protein to justify the assumption that this protein has a similar role is the TPRD protein (Tetratricopeptide repeat protein D; SWISS-PROT acc. no. P53804) which is also known as TTC3-HUMAN. This protein has a number of variations derived from alternative splicing, one of which is TPRDIII (EMBL acc. no. D84296). Until now, these proteins have had no function accorded to them. The sequences of TTC3_HUMAN and TPRDIII are given in FIGS. 3 and 4 herein.

The term “functional equivalent” is also intended to include alternatively spliced forms of the proteins of the invention. For example, TPRD (TTC3₁₃HUMAN) is known to exist in a number of differently-spliced forms and it is considered likely that the situation for FABLE will be similar, in that some or all of these alternatively spliced forms of FABLE will exhibit activity as tyrosine kinase modulator proteins. These alternatively-spliced forms are included as aspects of the present invention.

The term “functional equivalent” also refers to molecules that are structurally similar to the proteins of the present invention or that contain similar or identical tertiary structure. Such functional equivalents may be derived from the proteins of the present invention or they may be prepared synthetically or recombinantly using techniques of genetic engineering. In particular, synthetic molecules that are designed to mimic the tertiary structure or active site of the proteins of the present invention are considered to be functional equivalents as this term is used herein. For example, tyrosine kinase modulators of the present invention, such as FABLE, may also be used to provide the molecular basis for the design of small molecular compounds affecting the activity of the target tyrosine kinase. Variants and fragments of functional equivalents as defined above are themselves included in this aspect of the invention.

Derivatives of the proteins, variants and functional equivalents described above are also included as embodiments of the invention. Such derivatives may include one or more additional peptides fused at either or both of the amino- or carboxy-terminus of the proteins. The purpose of such peptides or polypeptides may be to aid detection, expression, separation or purification of the protein or to endow the protein with additional properties as desired. Examples of potential fusion partners include beta-galactosidase, luciferase, a polyhistidine tag, glutathione S transferase (GST) and a secretion signal peptide. Such derivatives may be prepared genetically or by chemically fusing the peptides or polypeptides.

According to a further aspect of the present invention, there is provided a ligand that binds to a protein, variant or functional equivalent thereof, as defined above. Such ligands may come in various forms, including natural or modified substrates, enzymes, receptors, small organic molecules such as small natural or synthetic organic molecules of up to 2000 Da, preferably 800Da or less, peptidomimetics, inorganic molecules, peptides, polypeptides, antibodies, structural or functional mimetics of these compounds. These ligands are likely to be useful in the diagnosis and treatment of mammalian diseases such as Abl-caused leukaemias, for example, by allowing the detection of aberrant levels or activities of the tyrosine kinase modulators described herein. Patients showing such abnormal levels would be potential candidates for preventative treatment or for frequent testing for Abl-related disease.

The ligands of this aspect of the invention may themselves act as tyrosine kinase modulators by binding to the tyrosine kinase modulator proteins described above and thus having a positive or negative effect on the activity of these proteins. Such a downstream modulatory effect may be through affecting the activity of the tyrosine kinase modulator, or may be by affecting its levels, by titrating out the levels of active protein in a cell or in systemic circulation.

In one embodiment of this aspect of the invention, the ligands are antibodies. The antibody or alternative ligand may be fused to a label, such as a radioactive, fluorescent, enzymatic, toxin or a secondary antibody label, in order to aid detection of FABLE and the variants and functional equivalent described herein.

Conveniently, the proteins, variants and functional equivalents of the invention may be prepared in recombinant form by expression in a host cell. Such expression methods are well known to those of skill in the art and many are described in detail by Sambrook et al (1989) and Fernandez & Hoeffler (1998).

According to a further aspect of the invention, there is provided a nucleic acid molecule encoding a protein, variant thereof or functional equivalent thereof according to the above-described aspects of the invention. Such molecules include single- or double-stranded DNA, cDNA and RNA, as well as synthetic nucleic acid species. Preferably, the nucleic acid species comprise DNA.

The invention also includes cloning and expression vectors containing the DNA sequences of this aspect of the invention. Such expression vectors may incorporate the appropriate transcriptional and translational control sequences, for example enhancer elements, promoter-operator regions, termination stop sequences, mRNA stability sequences, start and stop codons or ribosomal binding sites, linked in frame with the nucleic acid molecules of the invention.

Additionally, it may be convenient to cause a recombinant protein to be secreted from certain hosts. Accordingly, further components of such vectors may include nucleic acid sequences encoding any one of secretion, signalling and processing sequences.

Vectors according to the invention include plasmids and viruses (including both bacteriophage and eukaryotic viruses), as well as other linear or circular DNA carriers, such as those employing transposable elements or homologous recombination technology. Many such vectors and expression systems are known and documented in the art (Fernandez & Hoeffler, 1998). Particularly suitable viral vectors include baculovirus-, adenovirus- and vaccinia virus-based vectors.

Suitable hosts for recombinant expression include commonly-used prokaryotic species, such as E. coli, or eukaryotic yeasts that can be made to express high levels of recombinant proteins and that can easily be grown on large quantities. Mammalian cell lines grown in vitro are also suitable, particularly when using virus-driven expression systems. Another suitable expression system is the baculovirus expression system that involves the use of insect cells as hosts. An expression system may also constitute host cells that have the DNA incorporated into their genome. Proteins, or protein fragments may also be expressed in vivo, for example in insect larvae or in mammalian tissues.

A variety of techniques may be used to introduce the vectors according to the present invention into prokaryotic or eukaryotic cells. Suitable transformation or transfection techniques are described in the literature (Sambrook et al, 1989; Ausubel et al, 1991; Spector, Goldman & Leinwald, 1998). In eukaryotic cells, expression systems may either be transient (e.g. episomal) or permanent (chromosomal integration) according to the needs of the system.

Nucleic acid molecules according to the present invention may also be used to create transgenic animals, particularly rodent animals. This may be done locally by modification of somatic cells, or by germ line therapy to incorporate heritable modifications. Such transgenic animals may be particularly useful in the generation of animal models for drug molecules effective as ligands of the tyrosine kinase modulators that are described herein.

The invention therefore also includes transformed or transfected prokaryotic or eukaryotic host cells, or transgenic organisms containing a nucleic acid sequence as defined above. The invention also provides a method for screening for a compound effective to treat a disease or an abnormal physiological condition in which any of the above-described proteins are implicated, by contacting a non-human transgenic animal as described above with a candidate compound and determining the effect of the compound on the physiological state of the animal.

A further aspect of the present invention provides a method for preparing a protein, variant or functional equivalent, as defined above, which comprises culturing a host cell containing a nucleic acid molecule according to the invention under conditions whereby said protein is expressed and recovering said protein thus produced.

The invention also provides a pharmaceutical composition comprising a tyrosine kinase modulator, variant or functional equivalent according to the above-described aspects of the invention, in conjunction with a pharmaceutically acceptable carrier. Pharmaceutical compositions are also provided that comprise nucleic acid molecules encoding said tyrosine kinase modulators, variants or functional equivalents, as described previously. The invention also provides a pharmaceutical composition comprising a ligand according to the above-described aspects of the present invention in conjunction with a pharmaceutically acceptable carrier.

Pharmaceutically acceptable carriers include any carrier that does not itself induce the production of antibodies harmful to an individual receiving the composition. Suitable carriers are typically large, slowly metabolised molecules such as proteins, polysaccharides, polylactic acids, polyglycolic acids, polymeric amino acid, amino acid copolymers, lipid aggregates (such as oil droplets or liposomes) and inactive virus particles. Such carriers are well known to those of skill in the art.

According to a further aspect, the present invention provides for the use of the tyrosine kinase modulators, variants thereof, functional equivalents thereof, and of the ligands and nucleic acids according to any one of the aspects of the invention described above to modulate the activity of tyrosine kinases. Preferably, said tyrosine kinase modulators are used in animals, thereby to control the pathological effects of tyrosine kinases when aberrantly expressed or in the diagnosis of disease. Preferably, such animals are mammals, more preferably humans. According to a further aspect of this embodiment of the invention, there is provided the use of a functional equivalent of FABLE, such as TTC3₁₃HUMAN or TPRDIII, as a tyrosine kinase modulator.

The invention also provides a method of treating an animal suffering from a tyrosine kinase-mediated disease comprising administering to said animal an effective dose of a tyrosine kinase modulator, a variant thereof or a functional equivalent thereof, or a nucleic acid encoding said tyrosine kinase modulator as described above. The invention further provides a method of treating an animal suffering from a tyrosine kinase-mediated disease comprising administering to said animal an effective dose of a ligand according to the above-described aspects of the invention. Preferably, said animal is a mammal, most preferably it is human. Preferably, said disease is cancer. Most preferably, it is leukaemia.

The present invention also includes the use of the proteins and ligand as tools in the study of tyrosine kinase activity modulation and the physiological effects of such modulation including its role in diseases such as cancer.

The present invention also provides the use of a protein comprising the amino acid sequence given in FIG. 3, a variant thereof or a functional equivalent thereof as a modulator of tyrosine kinase activity. The invention further provides use of a protein consisting of the amino acid sequence given in either FIG. 3 or FIG. 4, or a variant or functional equivalent thereof as a modulator of tyrosine kinase activity.

The invention also provides methods for screening for small molecule drug ligands capable of interaction with the proteins, variants and functional equivalents described above. Such a method may involve any conventional method of high-throughput screening, as will be clear to the skilled reader.

For example, a suitable protein-based assay might involve contacting protein that is either free in solution, affixed to a solid support, borne on a cell surface or located intracellularly, with a test compound. Any response to the test compound, for example a binding response, or a stimulation or inhibition of a functional response may then be compared with a control where the protein or cells were not contacted with the test compound. One example of such a technique is a competitive drug screening assay, where neutralising antibodies that are capable of specifically binding to the protein compete with a test compound for binding. In this manner, the antibodies may be used to detect the presence of any test compound that possesses specific binding affinity for the protein. Alternative binding assay methods are well known in the art and include cross-linking assays and filter binding assays. The efficacy of binding may be measured using biophysical techniques including surface plasmon resonance and spectroscopy.

High throughput screening is a type of assay which enables a large number of compounds to be searched for any significant binding activity to the protein of interest (see, for example, WO84/03564). This is particularly useful in drug screening. In this scenario, many different small test compounds are synthesised onto a solid substrate. The protein is then introduced to this substrate and the whole apparatus washed. The protein is then immobilised by, for example, using non-neutralising antibodies. Bound protein may then be detected using methods that are well known in the art. Purified protein may also be coated directly onto plates for use in the aforementioned drug screening techniques.

Various aspects and embodiments of the present invention will now be described in more detail by way of example, with particular reference to the modification of the activity of the tyrosine kinase, Abl. It will be appreciated that modification of detail may be made without departing from the scope of the invention.

### BRIEF DESCRIPTION OF THE FIGURES

FIG. 1: Amino acid sequence of FABLE (one letter amino acid codon usage). Underlined are the N-terminal ‘Zn-finger-like structure’, the putative coiled coil domain, the proline stretch and the C-terminal RING-H2 finger.

FIG. 2: Amino acid sequence of SIA (one letter amino acid codon usage). Underlined are the putative coiled-coil domain, the proline stretch and the C-terminal RING-H2 finger.

FIG. 3: Amino acid sequence of TTC3_HUMAN (one letter amino acid codon usage).

FIG. 4: Amino acid sequence of TPRDIII (one letter amino acid codon usage).

FIG. 5: SIA is shown to counteract the lethal effect of c-Abl expression in S. pombe. Co-expression of c-Abl with HA-SIA restores exponential yeast growth and diminishes the amount of tyrosine phosphorylated proteins in the yeast cells.

FIG. 6: Schematic structures of FABLE and SIA showing positions of the ‘Zn-finger-like structure’, putative coiled coil, proline rich domain and RING-H2 finger domains.

FIG. 7: FABLE is a cellular form of SIA protein. Anti-SIA immunoprecipitation of 293 cell extracts shows that the cellular form of SIA has an apparent molecular weight of 160 kDa. Northern analysis shows that FABLE is ubiquitously expressed in human tissues.

FIG. 8: Association of Abl and. SIA in vivo. Abl and SIA co-immunoprecipitate from transfected 293 cells using anti-Abl or anti-SIA antibodies

FIG. 9A: FABLE and SIA bind to the SH3 domain and catalytic domains of c-Abl.

FIG. 9B: The putative coiled coil domain of FABLE and SIA is required for binding to Abl.

FIG. 10: SIA modulates the activity of Abl in 293 cells Co-expression of SIA with Abl PP (an activated form of Abl) enhances the activity of Abl and induces the phosphorylation of a protein of 72 kDa (pp72). Furthermore, expression of SIA activates c-Abl.

FIG. 11: FABLE activates c-Abl in 293 cells. Co-expression of FABLE with ΔSH3 Abl (an activated form of Abl) induces the phosphorylation of a protein of 72 kDa (pp72). Amino acids in the putative coiled-coil domain of FABLE (aa754-837) are required for the activation of c-Abl by FABLE. As shown on the anti-phosphotyrosine Western blot, deletion of these amino acids abolishes the activation of c-Abl.

FIG. 12: Summary of the ability to activate c-Abl by FABLE mutants. Amino acids in the putative coiled coil domain of FABLE (aa754837) are required for the activation of c-Abl by FABLE.

FIG. 13: Putative mechanism of FABLE action. FABLE binds to and activates c-Abl at particular subcellular sites. The activated c-Abl phosphorylates p72 that might also bind to FABLE, or FABLE might transport c-Abl to a new subcellular site where it can meet and phosphorylate p72.

### EXAMPLES

1) Cloning of SIA and FABLE

Yeast Strains and Culture Conditions

The Schizosaccharomyces pombe strain used was a derivative of SP813 (h^{-N} leul-32 ura4-D18 ade6-210). Strain G324 is SP813 carrying a stable version of pRSP-Abl-myr⁻ from which the autonomously replicating sequence ars1 had been deleted by digestions with SwaI and MluI. The pRSP-Abl-myr⁻ plasmid cannot be retrieved from strain G324. Growth conditions and media were as previously published (Superti-Furga et al., 1993).

DNA Constructs Relevant to the Yeast Experiments and Library Screening

pRSP and the pADH-X library expression vector have been described previously (Superti-Furga et al., 1996). Briefly, pADH-X was constructed from scratch starting from pSP73 to minimize size (Promega). The NdeI-XhoI fragment of pSP73 was replaced by a synthetic double-stranded oligonucleotide with ends compatible to, but not regenerating, NdeI and XhoI sites and containing the following restriction sites: HindIII, NruI, SpeI, NotI, SmaI and EcoRI to obtain pSP73-RB. The S. pombe ura4 gene was cut out of pAU and cloned into the HindIII site of pSP73-RB. The S. pombe ars1 was cloned out of pRSP and into the EcoRI site. A short version (357 bp) of the polyadenylation site of the nmt1 was recovered from plasmid pREP1 (Maundrell, 1993) with AvaI, blunted with Klenow polymerase and cloned into the SmaI site of the pSP73-RB polylinker to obtain pPLV. The promoter from the S. pombe adh1 gene was isolated as a HindIII-BamHI fragment from pAU, blunt-ended and cloned into the NruI site of pPLV to obtain pPLV-Adh. By so doing, the BamHI site was regenerated. A synthetic double-stranded oligonucleotide was inserted at the NotI site. The oligonucleotide introduced the following sites: BstXI, XhoI, NruI, NdeI, BstXI and BamHI. The two BstXI sites were the same asymmetrical sites used in CDM8 (Seed, 1987), incapable of self-ligation. The resulting plasmid was called pADH-X (where X stands for expression).

pRSP-Abl-myr⁻ was prepared by cloning an adaptor oligonucleotide bearing the G2A mutation into pRSP-c-Abl (Walkenhorst et al., 1996) cut with XhoI and StuI. The adapter sequence was:

5′-TCGACCATGGCGCAGCAGCCTGGAAAAGTTCTTGGGGACCAAAG-AAGG-3′ (upper strand oligonucleotide) and 5′-CCTTCTTTGGTCCCCAAGAACTTTTCCAGGCTGCTGCGCCATGG-3′ (lower strand oligonucleotide).

cDNA Libraries

PolyA⁺ RNA from SV40 large T-transformed primary human lung fibroblasts IMR-90 (a kind gift of R. Pepperkok, University of Geneva) was obtained from total RNA using oligotex-dT (Qiagen). 10 μg of polyA⁺ RNA was transformed into first strand cDNA using the Superscript retrotranscriptase II (BRL) and converted to double stranded cDNA by the Gubler and Hoffmann method (Gubler and Hoffmann, 1983) using BRL enzymes. The cDNA was ligated to excess BstXI adapters (Invitrogen) and size-selected for products of 900 bp in length or more on an agarose gel and recovered using glass-milk beads (Geneclean, BIO101). The cDNA was cloned in pADH-X digested with BstXI. Electroporation of INVαF′ E. coli cells (Invitrogen) yielded 4×10⁶ independent clones. cDNA obtained from the Burkitt lymphoma cell line BJA-B and ligated to BstXI adapters was a kind gift of Dr. Meinrad Busslinger (IMP, Vienna). The BJA-B cDNA was cloned in pADH-X and used to generate a library of 1×10⁶ independent clones.

Yeast Transformation and Library Screening for cDNAs Able to Counteract the Lethal Effect of c-Abl

Transformation of S. pombe was done by the lithium acetate (c) method as described (Moreno et al., 1991; Superti-Furga et al., 1993). 650 μg fibroblast library DNA was used: to transform 2.2×10¹⁰ G324 S. pombe cells. An aliquot of the transformation mixture was plated on PMA plates containing thiamine to test the transformation efficiency (2×10⁷ transformants in total). The transformation mixture was plated on twenty 24×24 cm PMA plates with thiamine and incubated at 30° C. Three days after plating, approximately 1×10⁶ colonies from each plate were collected using approximately 20 ml of PMA/thiamine and a rubber policeman. The cells from the twenty plates were collected, pooled, mixed extensively and washed once with PMA (no thiamine). The cell pellet was resuspended in YEA/20% glycerol and frozen at −80° C. in aliquotes. The titer of colony-forming cells was determined. The equivalent of 8×10⁷ cells were thawed, washed three times with PMA to remove thiamine and put into culture in a shaking water bath for 7 h at 30° C. Cells were finally plated onto ten 24×24 cm PMA plates with no thiamine. Growing colonies were picked 4-6 days after. Out of more than 2000 colonies of all sizes, 100 were picked that represented all size classes and streaked on PMA plates with no thiamine.

Plasmid Retrieval

Plasmids were retrieved from the S. pombe colonies by the following method. Cell pellets from 2 ml liquid culture or from a fresh plate were resuspended in 200 μl disruption buffer (100 mM NaCl, 10 mM Tris-HCl pH 8.0, 1 mM EDTA, 2% Triton X-100, 1% SDS). 200 μl phenol:chloroform (1:1) and 300 μl glass beads were added to the resuspended pellet followed by 2 minutes vortexing on a multi-mix in the cold. Phases were separated by a 5 minute-centrifugation at full speed in a microfuge. The aqueous phase was re-extracted with phenol:chloroform (1:1) and precipitated with ethanol after addition of sodium acetate to 0.3 M. The pellet was washed and resuspended in 30 μl H₂O. 5 μl were used to electroporate Escherichia coli XL1-Blue cells. The plasmids of two bacterial colonies were analyzed from each S. pombe colony. The plasmids were characterized by BamHI or NotI digestion followed by agarose gel electrophoresis. Both enzymes have two sites each at the 5′ and 3′ sides of the cDNA insert cloning site. From about 10% of the colonies it was impossible to retrieve any plasmids. Of the remaining colonies, about 10% yielded plasmids with no insert. Approximately 10% of the remaining colonies rearranged plasmids of very high molecular weight. Approximately a third of the other plasmids gave two or more inserts using NotI or BamHI, and a normal plasmid backbone size, suggesting multiple inserts. In these cases, the inserts were also subcloned individually in pADH-X for retransformation of S. pombe.

Further Testing of Plasmids Retrieved

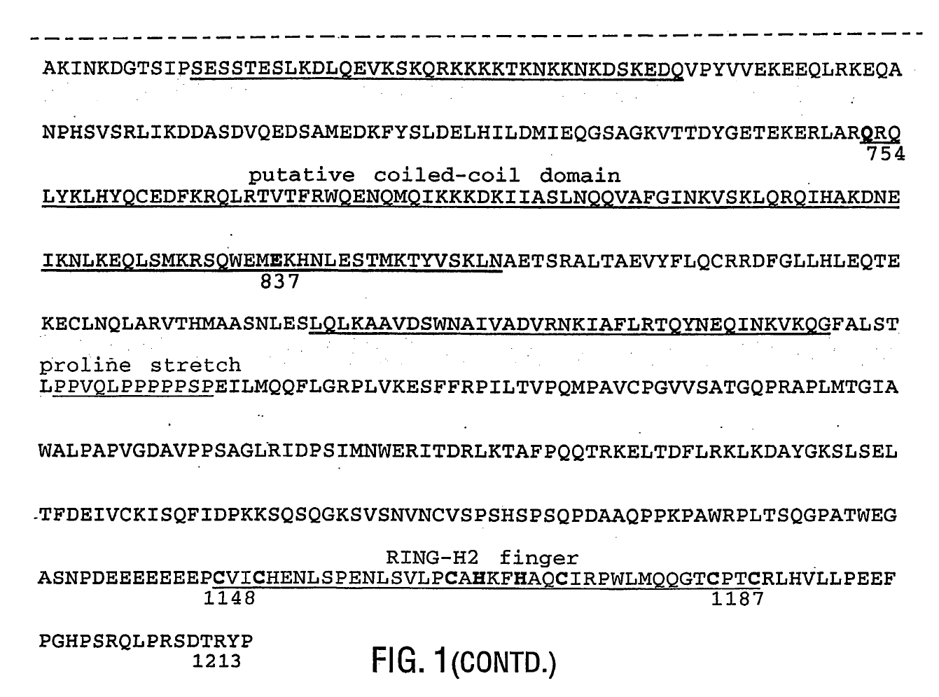

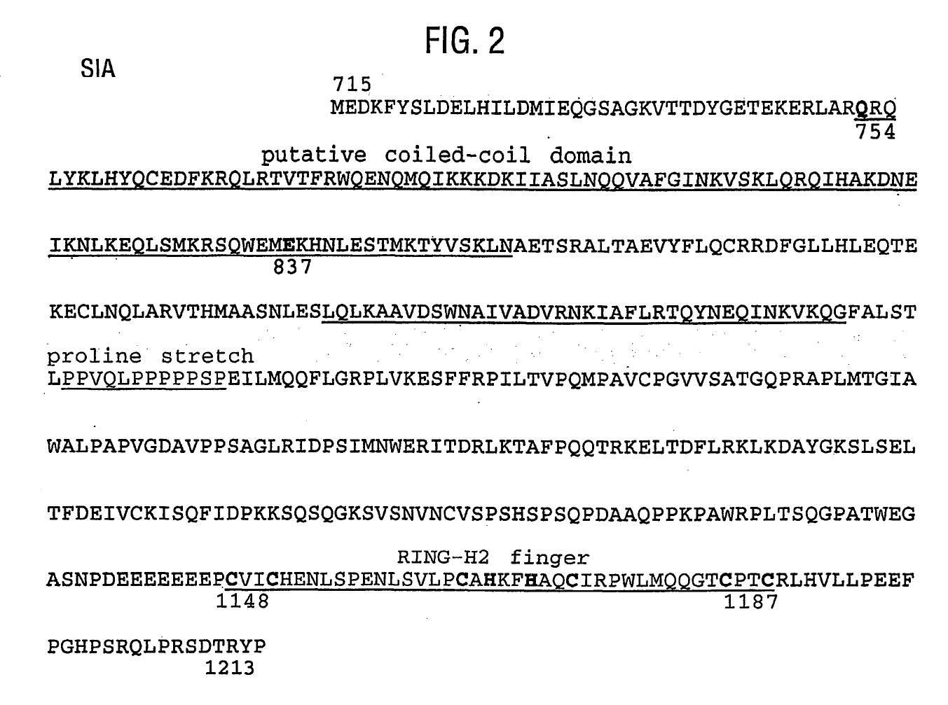

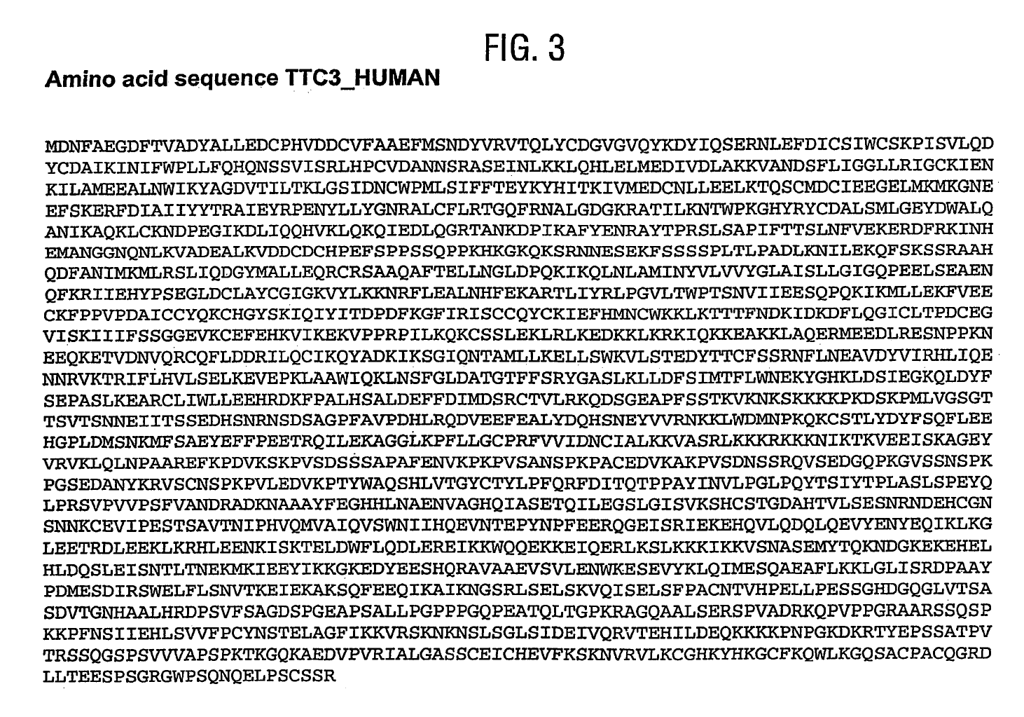

To test if the ability to antagonize the Abl-induced growth inhibition was a plasmid-bound property, the plasmids were retransformed in the original strain and plated both on plates with or without thiamine. Some clones grew as well or better on plates without thiamine. Colonies growing from the plates with thiamine were streaked individually and replica plated on PMA plates without thiamine. Only 16 plasmids were able to antagonize the Abl-induced growth inhibition. Sequence analysis and restriction patterns revealed that the 16 plasmids actually represented 10 different classes, as one clone had three related sequences and two had two. The 10 plasmids were also tested in a strain that contained the inducible Src tyrosine kinase gene (Superti-Furga et al., 1996). Several plasmids antagonized Src-induced growth inhibition to very different extents. Only two clones, #37 and #113, did not confer any growth to the Src strain. Here, only #113, which we refer to as SIA (Sequence Inhibiting Abl), will be discussed further. pADH-X-SIA contains an insert of 2278 bp, coding for a protein of 499 amino acids (SIA). The amino acid sequence of SIA is given in FIG. 2.

S. pombe Cell Lysates

Native protein lysates from S. pombe cells were done by the lysis buffer-glass beads method (Superti-Furga et al., 1993) or denatured extracts were obtained by directly boiling the cells in SDS-containing loading buffer. 20-50 μg of extracts were analyzed by SDS-PAGE followed by immunoblotting.

Subcloning of SIA DNA

pADH-X-SIA (the original clone retrieved from the library screening) was digested with BamHI and the released insert was cloned into the BamHI site of vector pBluescriptII-KS⁻ (Stratagene) giving rise to pBS-SIA. Via PCR amplification of pBS-SIA DNA using oligonucleotides 163 (AATTAGGATCCATGGAAGACAAGTTCTATAG) and 164 (AATTA-GGATCCTCATGGGTACGMIGTATC) BamHI sites were introduced directly in front of and behind the SIA open reading frame (ORF). Digestion of the PCR product with BamHI and subcloning into vector pAHA (a derivative of pADH-X containing an influenza virus hemagglutinin (HA) tag sequence) digested with BamHI resulted in plasmid pAHA-SIA^{ORF}. In this way, the ORF of SIA was cloned in frame behind the HA tag of vector pAHA giving rise to HA-SIA upon expression in yeast.

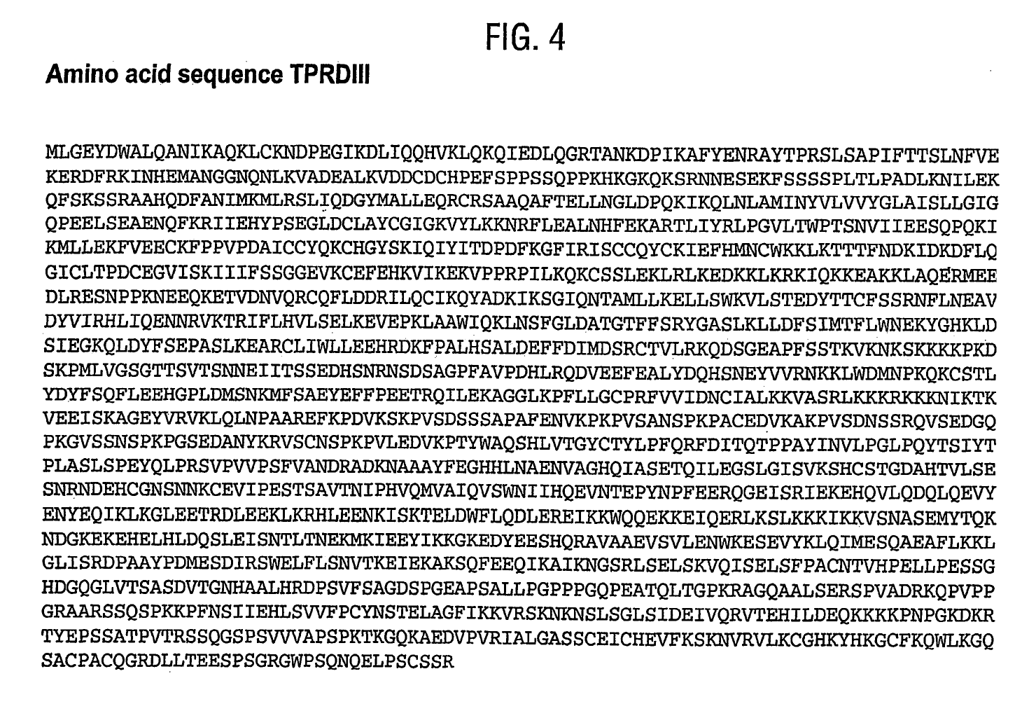

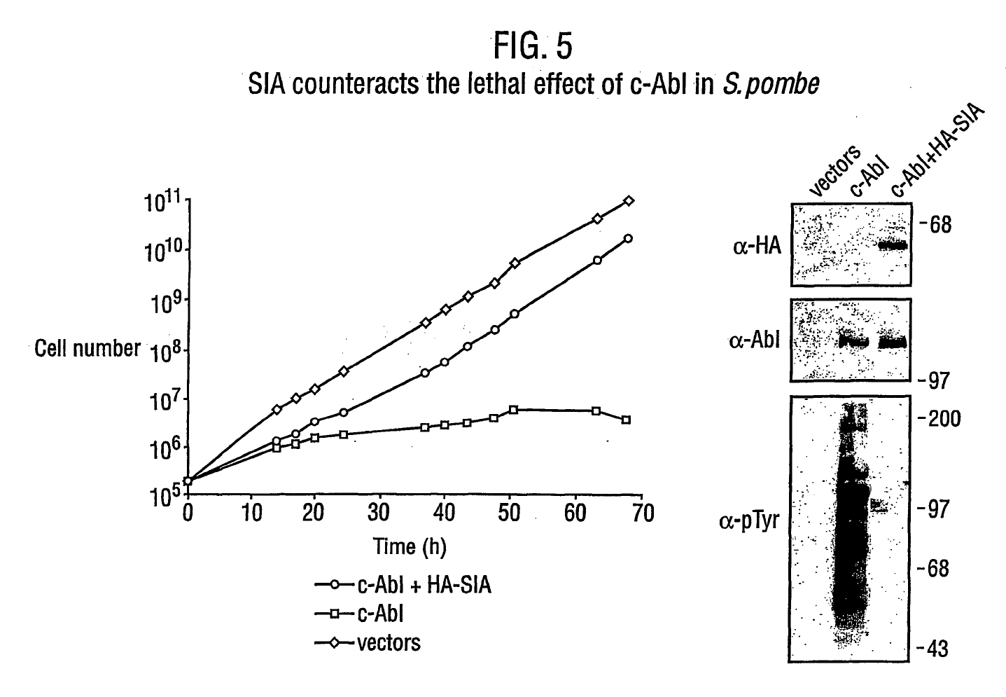

Co-expression of c-Abl with HA-SIA was shown to restore exponential yeast growth and diminish the amount of tyrosine phosphorylated protein in yeast cell (FIG. 5).

For in vitro translation and expression in mammalian cells, the SIA ORF was digested from pAHA-SIA^{ORF} with BamHI and subcloned into vector pcHA digested with BamHI, resulting in pcHA-SIA. Vector pcHA was generated by inserting the SalI (made blunt with Klenow enzyme)-NotI fragment containing the HA tag sequence from pAHA into the HindIII (made blunt with Klenow enzyme)/NotI sites of vector pcDNA3 (Invitrogen). The BamHI insert of pAHA-SIA^{ORF} was also subcloned into the BamHI site of mammalian expression vector pEBG (Tanaka et al., 1995) and E. coli expression vector pGEX-2T (Amersham Pharmacia), giving rise to pEBG-SIA and pGEX-SIA, respectively.

Cloning of Full-Length FABLE cDNA

Full-length FABLE cDNA was retrieved by screening of a human Lymph Node cDNA library in λgt11 (Clontech). A 650 bp NcoI-Bsu36I SIA DNA fragment from plasmid pAHA-SIA^{ORF} was random primed using Klenow enzyme and ³²P-dCTP (Amersham Pharmacia) and used to probe the cDNA library following standard procedures (Ausubel et al., 1987). Phage DNA from positive clones was isolated, digested with EcoRI subcloned into the EcoRI site of vector pBluescriptII-KS⁻ and sequenced. In this way, one clone was retrieved (#13, pBS-13) that consisted of at least 440 bp of SIA sequence and 2271 bp of sequence extending further to the 5′ end, comprising the full-length FABLE cDNA.

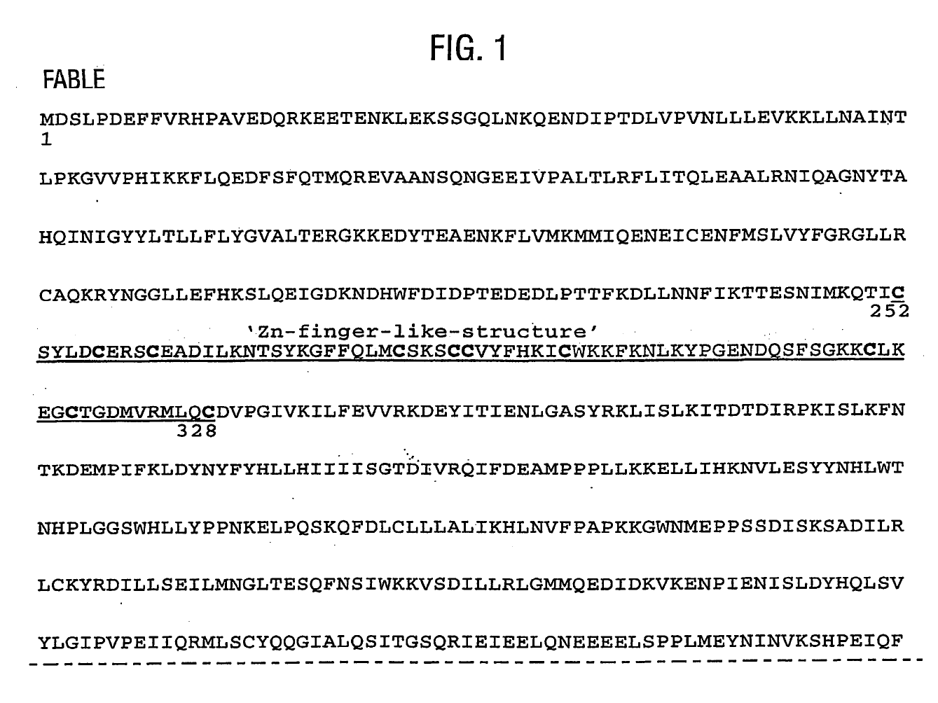

To assemble the full-length FABLE cDNA, pBS-SIA was digested with XhoI and BglII (partial) releasing a fragment of 1900 bp containing the 3′ end of FABLE cDNA. pBS-13; was digested with Asp718 and BglII (partial) giving rise to a fragment of 2700 bp consisting of the 5′ end of FABLE cDNA. Both fragments were ligated into vectors pBluescriptII-KS⁻ and pcDNA3 digested with Asp718 and XhoI, giving rise to full-length pBS-FABLE and pcDNA-FABLE. The full-length FABLE cDNA consists of 4533 bp coding for a protein of 1213 amino acids. The amino acid sequence of FABLE is given in FIG. 1.

Subcloning of FABLE DNA

The following oligonucleotides were used for the subcloning of FABLE DNA:

164: AATTAGGATCCTCATGGGTACCTTGTATC
198: GAGCCTTTTGTGATCTGTCAT
199: ATGACAGATCACAAAAGGCTC
204: TGGGGATCCGAAAAACATAATCTGGAAAGC
296: AAGGATCCGCCACCATGGATTCTCTACCAGATGAATTTTTTG
310: CAATACCCAGTGAATCTTCAACAG
311: ATTATGTTTACGAGCAAGCCTTTCCTTTTCAG
312: CTTGCTCGTAAACATAATCTGGAAAGCACAATG

For transient transfection in mammalian cells FABLE DNA was subcloned to the mammalian expression vector pEBB (Tanaka et al., 1995). The original FABLE

cDNA was mutagenised by PCR using oligonucleotides 296/164 to obtain a favourable startcodon context (Kozak sequence) (Kozak, 1987; Kozak, 1992). The resulting PCR product containing the full-length FABLE DNA was digested with BamHI and subcloned into the BamHI site of pEBB. The FABLE point mutation C1148F was generated by two step PCR mutagenesis using oligonucleotides 204/199 and 198/164 in the first PCR step, and 204/164 in the second PCR step. The PCR product was digested with Bsu36I and subcloned into the full-length pEBB-FABLE construct which was digested with SpeI (made blunt with Klenow enzyme) and Bsu36I, giving rise to pEBB-FABLEC1148F. The deletion Q754-E837 was obtained by two step PCR mutagenesis using oligonucleotides 310/311 and 312/193 for the first PCR step, and 310/193 for the second PCR step. The PCR product containing the Q754-E837 deletion was cloned into full-length FABLE DNA using NdeI and Bsu36I, resulting in pEBB-FABLEΔQ754-E837.

For analysis in S. pombe, FABLE DNA was subcloned into pADH-X and pRSP. pBS-FABLE was digested with NotI and the released FABLE insert was ligated into pADH-X and pRSP digested with NotI.

Anti-FABLE Antibody

Polyclonal anti-FABLE rabbit antibody was generated by immunizing rabbits with 100-300 μg bacterially expressed purified GST-SIA fusion protein. All immunizations and handling of the rabbits was done by the EMBL animal facility.

GST-SIA Constructs

The following oligonucleotides were used for the GST-SIA constructs:

163: AATTAGGATCCATGGAAGACAAGTTCTATAG
193: GCGAATTGTTACTTGACTTTGTTTATTTGTTCA
194: CAAAGGATCCCAAGGATTTGCCTTGAGTAC
195: GGGAATTCTTATCCTTCCCATGTGGCAGGAC
196: TGGGGATCCGCCAGTAATCCAGATGAGGA
197: ATGAATTCATGGGTACCTTGTATCAG
198: GAGCGTTTTGTGATGTGTCAT
199: ATGACAGATCACAAAAGGCTC
204: TGGGGATCCGAAAAACATAATCTGGAAAGC
205: TCGAATTCTTATTTTCCATAAGCATCCTTCAA
222: ACTGGATCGTGGCAAGAAAACCAAATGCAG
223: AGGAATTCTTATAAGAACTGCTGCATCAGTAT
224: ATGAATTCTTAATTCATGATGGAGGGATCAA

GST-SIA constructs were obtained by PCR amplification of PADH-X-SIA using oligonucleotides 163/193, 194/195, 196/197, 163/195, 194/197, 163/205, 204/195, 204/205, 163/223, 163/224, 222/205, 222/223 and 222/224 giving rise to SIA1-224, SIA225-420, SIA421-499, SIA1-420, SIA225-499, SIA1-353, SIA121-420, SIA121-353, SIA1-252, SIA1-319, SIA64-353, SIA 64-252 and SIA64-319, respectively. The PCR products were digested with BamHI/EcoRI and cloned into vector pGEX-2T.

pGEX-SIAC434F was obtained by a two step PCR reaction using oligonucleotides 197/198, 163/199 and pADH-X-SIA as template in the first PCR amplification step. The resulting PCR products were purified and used as template in the second PCR reaction using oligonucleotides 163/197. The resulting PCR product containing the C434F mutation was digested with BamHI/EcoRI and cloned into pGEX-2T.

Expression and Purification of GST-SIA Fusion Protein

GST-SIA fusion protein was expressed from plasmid pGEX-SIA in E. coli Xl1-Blue. Xl1-Blue containing pGEX-SIA was grown at 37° C. until OD600 reached 0.8 and then expression of fusion protein was induced for 4 h at 30° C. with 0.1 mM IPTG. Bacteria (50 ml culture) were collected by centrifugation (10′, 4000 rpm, 4° C.), washed with STE (10 mM Tris-HCl pH 8, 150 mM NaCl, 1 mM EDTA) and resuspended in 3 ml cold 0.1 mg/ml lysozyme/STE. The suspension was incubated for 15′ on ice, DTT and protease inhibitors were added (5 mM DTT, 1 mM PMSF, 10 μg/ml TPCK, 5 μg/ml TLCK, 1 μg/ml leupeptin, 1 μg/ml aprotinin, 10 μg/ml soybean trypsin inhibitor) and 0.45 ml of 10% N-laurylsarcosine/STE was added to reach a final concentration of 1.5%. The suspension was vortexed for 5″, sonicated on ice (Branson sonifier, 3 times 6 pulses, 50% duty cycle, output level 5) and centrifuged (10′, 10,000 rpm, 4° C.). Triton-X100 was added to the supernatant (2% final concentration) and the crude extracts containing GST-SIA fusion protein were stored at −70° C.

GST-SIA was further purified by adding 200-300 μl Glutathion Sepharose beads (Amersham Pharmacia) to the crude extract and incubation for 1 h at 4° C. Beads were washed 3 times with buffer A (50 mM Tris-HCl pH 7.5, 150 mM NaCl, 1% NP40, 5 mM EDTA, 5 mM EGTA, 25 mM NaF, 1 mM orthovanadate, 1 mM PMSF, 10 μg/ml TPCK, 5 μg/ml TLCK, 1 μg/ml leupeptine, 1 μg/ml aprotinine, 10 μg/ml soybean trypsin inhibitor) and GST-SIA protein was eluted from the beads with 10 mM glutathion/50 mM Tris-HCl pH 8. GST-SIA was dialyzed against 15 mM Hepes pH 7.3, 50 mM NaCl, 0.25 mM DIT and concentrated using a Centricon-10 column (Amicon).

GST Pull Down

SIA, FABLE and c-Abl proteins were in vitro translated using the Coupled Reticulocyte Lysate System (Promega). One jig of SIA, FABLE or Abl DNA (pBS-SIA/pcHA-SIA, pcDNA-FABLE or pSGT-c-Abl (Barilá and Superti-Furga, 1998)) was incubated with TnT rabbit reticulocyte lysate and ³⁵S-methionine (Amersham Pharmacia) for 90′ at 30° C. Glutathion Sepharose beads were washed with 1% Triton-X100/PBS, incubated with GST protein or GST-Abl fusion protein (GST-SH3 Abl, GST-SL Abl, GST-IL Abl, GST-CD Abl, GST-SH2-SH3-CD Abl) for 1 h at 4° C. in 1% Triton-X100/PBS and washed twice with buffer A (50 mM Tris-HCl pH 7.5, 150 mM NaCl, 1% NP-40, 5 mM EDTA, 5 mM EGTA, 25 mM NaF, 1 mM orthovanadate, 1 mM PMSF, 10 μg/ml TPCK, 5 μg/ml TLCK, 1 μg/ml leupeptine, 1 μg/ml aprotinine, 10 μg/ml soybean trypsin inhibitor). In vitro translated SIA or FABLE protein was precleared by incubation with GST protein coupled to Glutathion Sepharose beads for 30′ at 4° C. in buffer A. The suspension was centrifuged and the supernatant incubated with GST-Abl fusion protein coupled to Glutathion Sepharose beads for 1 h at 4° C. in buffer. A. Beads were washed 3 times with buffer A and resuspended in SDS-sample buffer. Bound proteins were analyzed by SDS-PAGE.

The binding of GST-SIA protein to c-Abl was performed in a similar way. In short, GST-SIA protein (wt and deletion mutants) was coupled to Glutathion Sepharose beads as described above. In vitro translated c-Abl protein was precleared by incubation with GST protein and subsequently incubated with GST-SIA protein in buffer A or NETN (20 mM Tris-HCl pH 8, 100 mM NaCl, 1 mM EDTA, 0.5% NP-40). Beads were washed 3 times with buffer A or NETN and resuspended in SDS-sample buffer.

2) SIA and FABLE Modulate c-Abl Activity in 293 Cells

Transient Transfection and Preparation of Cell Extract

Human 293 embryonic kidney cells (293) were cultured in Dulbecco's Modified Eagle's Medium supplemented with 10% fetal calf serum. 293 cells were transfected with pEBG-SIA, pEBB-FABLE and/or Abl DNAs (wt and mutant Abl alleles in vector pSGT (Barilá and Superti-Furga, 1998)) using the calcium phosphate method (Ausubel et al., 1987). Forty to. 48 h after transfection the cells were washed once with PBS and lysed in buffer A (50 mM Tris-HCl pH 7.5, 150 mM NaCl, 1% NP40, 5 mM EDTA, 5 mM EGTA, 25 mM NaF, 1 mM orthovanadate, 1 mM PMSF, 10 μg/ml TPCK, 5 μg/ml TLCK, 1 μg/ml leupeptine, 1 μg/ml aprotinine, 10 μg/ml soybean trypsin inhibitor) for 10′ on ice. Insoluble material was pelleted (10′, 13,000 rpm, 4° C.) and the supernatant was used for immunoprecipitation and/or SDS-PAGE followed by immunoblotting. The total protein content of the extract was measured by Bradford protein assay (Bio-Rad).

Immunoblotting

Forty μg of total protein was separated by SDS-PAGE, blotted to nitrocellulose and probed with specific antibodies. The following antibodies were used: mouse monoclonal anti-Abl antibody Ab-3 (Oncogene Sciences, 1:500 in 3% BSA/PBS/0.1% Tween20), rabbit polyclonal anti-FABLE antibody (1:5000 in 5% milk/PBS/0.1% Tween20), mouse monoclonal anti-phosphotyrosine antibody 4G10 (Upstate Biotechnology, 1:2000 in 3% BSA/PBS/0.1% Tween20), mouse monoclonal anti-Tubulin antibody (Sigma, 1:7000 in 3% BSA/PBS/0.1% Tween20), mouse monoclonal anti-Src antibody 2-17 (1:2000 in 5% milk/PBS/0.1% Tween20). Detection was performed by incubation with horseradish peroxidase coupled secondary antibodies and the enhanced chemiluminescence western blot detection system (Amersham Pharmacia).

Immunoprecipitation

Abl or FABLE protein were immunoprecipitated from 400-1000 μg of total protein extract in buffer A (50 mM Tris-HCl pH 7.5, 150 mM NaCl, 1% NP-40, 5 mM EDTA, 5 mM EGTA, 25 mM NaF, 1 mM orthovanadate, 1 mM PMSF, 10 μg/ml TPCK, 5 μg/ml TLCK, 1 μg/ml leupeptine, 1 μg/ml aprotinine, 10 μg/ml soybean trypsin inhibitor) using 5 μl mouse monoclonal anti-Abl antibody (Ab-3, Oncogene Sciences) or 5 μl rabbit polyclonal anti-FABLE antibody. Immunecomplexes were recovered using protein G-Sepharose or protein A-Sepharose beads (Amersham Pharmacia) according to the first antibody preference. Beads were washed 3 times with buffer A and resuspended in SDS-sample buffer. Bound proteins were analyzed by 7.5% SDS-PAGE followed by immunoblotting.

Immunofluorescence

NIH-3T3 or HeLa cells were grown on coverslips and transfected with SIA or FABLE DNA using the calcium phosphate method. After 48 h the cells were washed with PBS and fixed with MeOH/Acetone or 3% paraformaldehyde/PBS. SIA/FABLE protein was detected by indirect immunofluorescence using rabbit anti-FABLE antibody diluted 1:50 in 5% goat serum/PBS, followed by incubation with FITC-goat-anti-rabbit antibody (Jackson Laboratories) diluted 1:50 in 5% goat serum/PBS.

Results

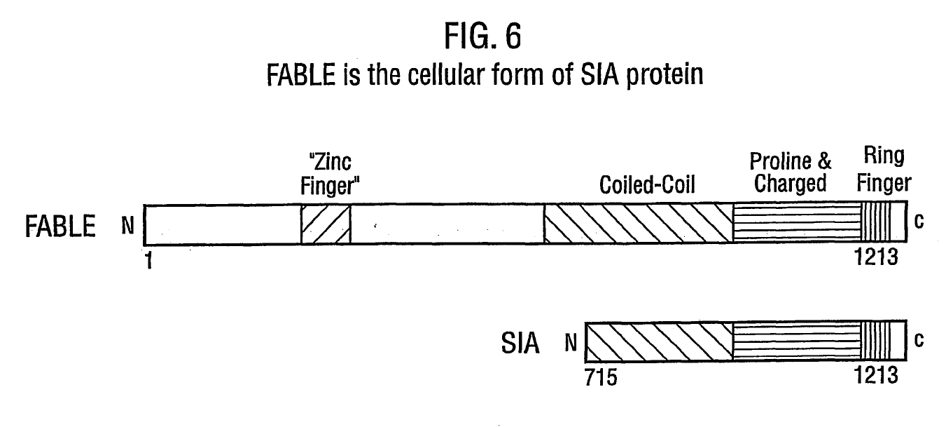

FABLE was shown to be the cellular form of SIA protein. Anti-SIA immunoprecipitation of 293 cell extracts showed that the cellular form of SIA has an apparent molecular weight of 160 kDa. Northern analysis demonstrated that FABLE is ubiquitously expressed in human tissues (FIG. 7).

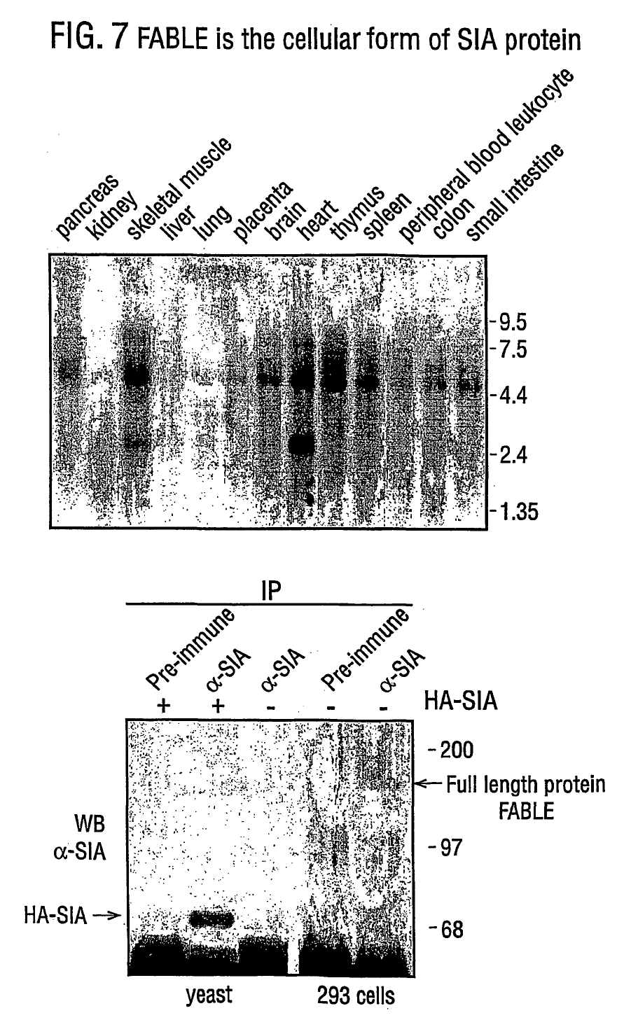

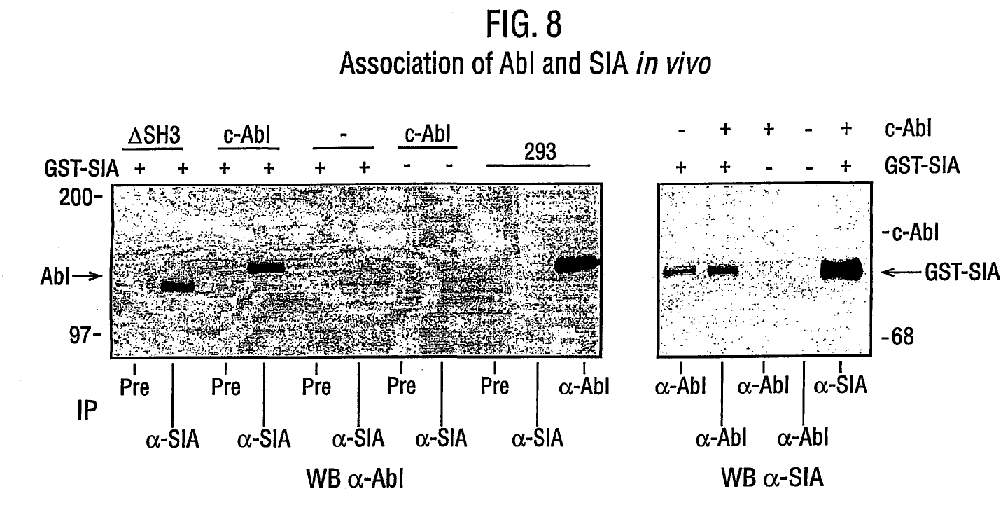

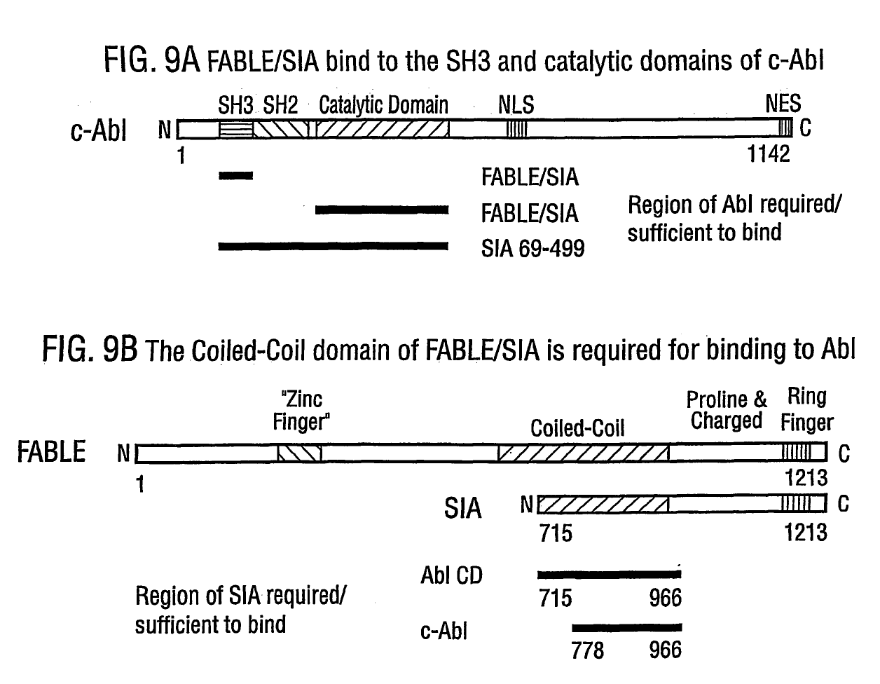

SIA was found to be associated with Abl in vivo. Abl and SIA co-immunoprecipitate from transfected 293 cells using anti-Abl or anti-SIA antibodies (FIG. 8). Furthermore, SIA was shown to modulate the activity of Abl in 293 cells. Co-expression of SIA with Abl PP, an activated form of Abl, enhanced the activity of Abl and induced phosphorylation of a protein of 72 kDa. Expression of SIA was also found to activate c-Abl (FIG. 10).

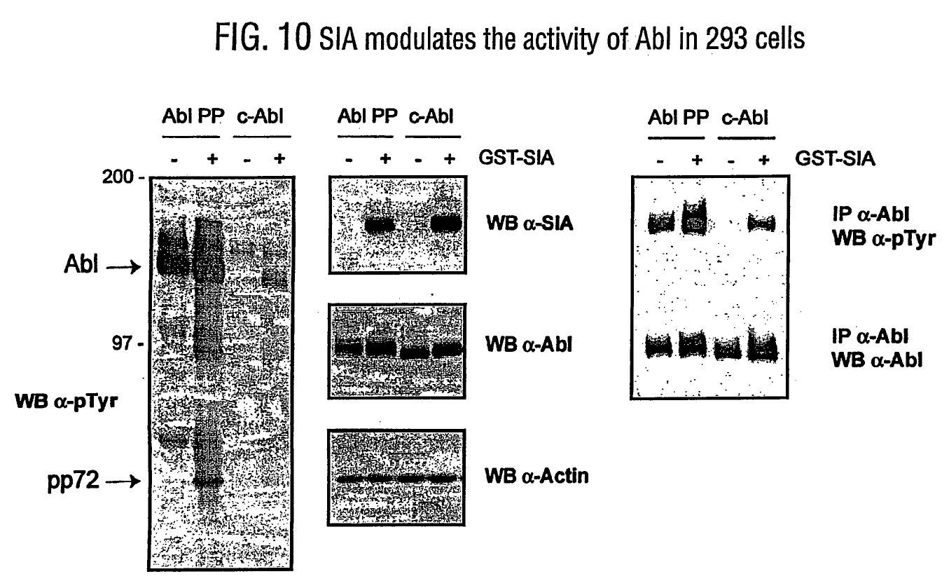

FABLE was also shown to modulate the activity of c-Abl in 293 cells. Co-expression of FABLE with ΔSH3 Abl, an activated form of Abl, induced phophorylation of the 72 kDa protein (FIG. 11). Amino acids in the coiled coil domain of FABLE appear to be required for activation since deletion of these amino acids abolishes activation of c-Abl by FABLE.

3) Conclusions

SIA and its cellular counterpart FABLE have been demonstrated to be modulators of the tyrosine kinase c-Abl. These proteins may also modulate the activity of other tyrosine kinases.

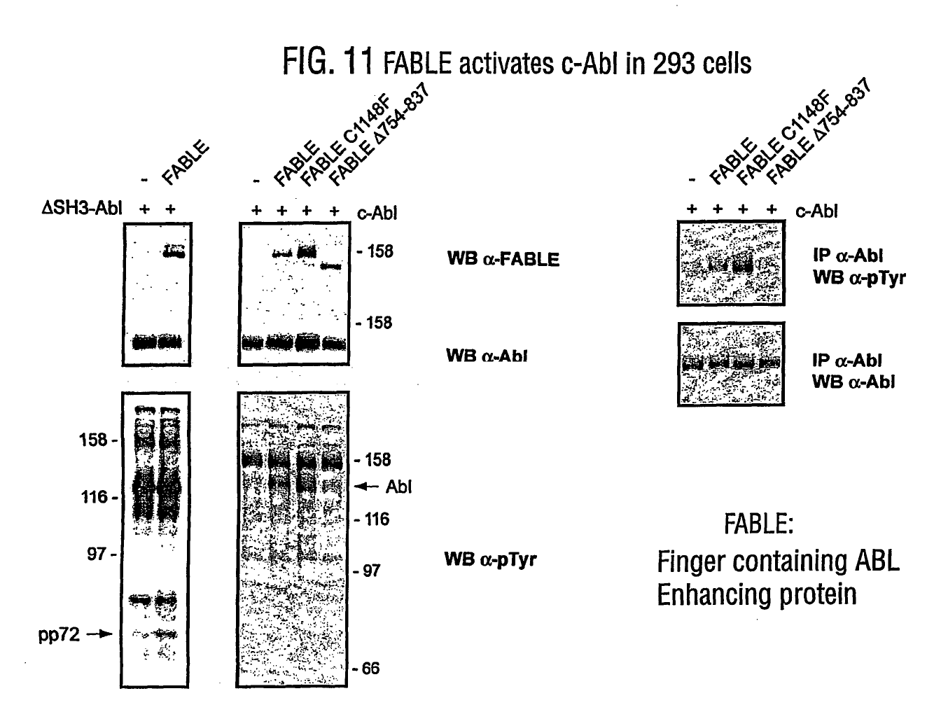

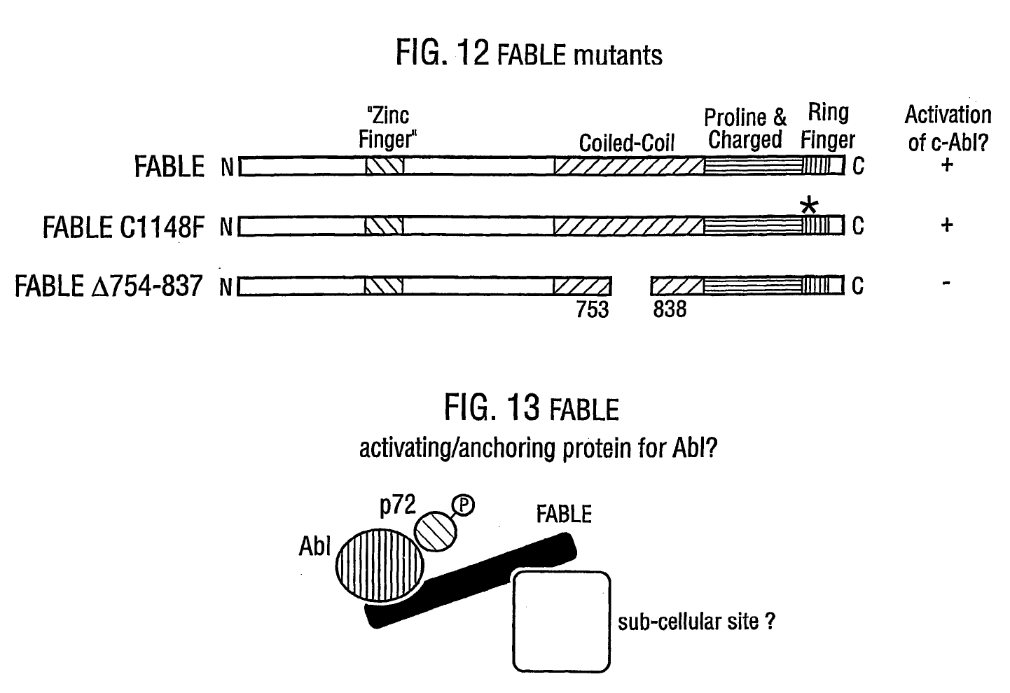

FIG. 12 provides a summary of the ability of FABLE mutants to activate c-AM. It appears that FABLE binds to the SH3 and catalytic domains of c-Abl and that the coiled coil domain of FABLE is required for this activation (FIG. 12). A possible mechanism of c-Abl activation by FABLE is set out in FIG. 13. FABLE binds to and activates c-Abl at particular subcellular sites. The activated Abl phosphorylates the 72 kDa protein, that might also bind FABLE. Alternatively, FABLE might transport c-Abl to a new subcellular site where it phosphorylates the 72 kDa protein.

### REFERENCES

Ausubel, F. M., Brent, R., Kingston, R. E., Moore, D. D., Seidman, J. G., Smith, J. A. and Struhl, K. (1987) Current protocols in molecular biology. John Wiley and Sons, New York.

Ausubel E. A. et al Current protocols in Molecular Biology, Wiley Interscience, New York. Fernandez J. M. & Hoeffler J. P., eds. (1998) Gene expression systems. Using nature for the art of expression. Academic Press, San Diego, London, Boston, New York, Sydney, Tokyo, Toronto.

Barilá, D. and Superti-Furga, G. (1998) An intramolecular SH3-domain interaction regulates c-Abl activity. Nature Genet., 18, 280-282.

Gubler, U. and Hoffmann, B. J. (1983) A simple and very efficient method for generating cDNA libraries. Gene, 25, 263-269.

Kozak, M. (1987) At least six nucleotides preceding the AUG initiator codon enhance translation in mammalian cells. J Mol Biol, 196, 947-950.

Kozak, M. (1992) Regulation of translation in eukaryotic systems. Annu Rev Cell Biol, 8, 197-225.

Maundrell, K. (1993) Thiamine-repressible expression vectors pREP and pRIP for fission yeast. Gene, 123, 127-130.

Mayer & Baltimore (1994) Mutagenic analysis of the roles of SH2 and SH3 domains in regulation of Abl tyrosine kinase. Molecular & Cellular Biology 14: 2883-2894

Moreno, S., Klar, A. and Nurse, P. (1991) Molecular genetic analysis of Fission Yeast Schizosaccharomyces pombe. In Guthrie, C. and Fink, G. (eds.), Guide to Yeast Genetics and Molecular Biology, Vol. 194, pp. 795-826.

Sambrook J. et al (1989) Molecular cloning: a laboratory manual. New York. Cold Spring Harbour Laboratory Press.

Sawyers C. L. (1992) The bcr-abl gene in chronic myelogenous leukaemia. Cancer Surveys 15: 37-51

Seed, B. (1987) An LPA-3 cDNA encodes a phospholipid-linked membrane protein homologous to its receptor CD2. Nature, 329, 840-842.

Spector et al (1998) Cells, a laboratory, manual. Cold Spring Harbour Press.

Superti-Furga, G., Fumagalli, S., Koegl, M., Courtneidge, S. A. and Draetta, G. (1993) Csk inhibition of Src activity requires both the SH2 and SH3 domains of Src. EMBO J., 12, 2625-2634.

Superti-Furga, G., Jönsson, K and Courtneidge, S. A. (1996) A functional screen in yeast for regulators and antagonizers of heterologous protein tyrosine kinases. Nature Biotechnology, 14, 600-605.

Tanaka, M., Gupta, R. and Mayer, B. J. (1995) Differential inhibition of signaling pathways by dominant-negative SH2/SH3 adapter proteins. Mol. Cell. Biol., 15, 6829-6837.

Van Etten R. A. (1999) Cycling, stressed-out and nervous: cellular function of c-Abl. Trends in Cell Biology 9: 179-186.

Walkenhorst, J., Goga, A., Witte, O. N. and Superti-Furga, G. (1996) Analysis of human c-Abl tyrosine kinase activity and regulation in S. pombe. Oncogene, 12, 1513-1520.

1. A tyrosine kinase modulator protein comprising the amino acid sequence given in FIG. 1, a variant thereof or a functional equivalent thereof.

2. A tyrosine kinase modulator protein consisting of the amino acid sequence given in FIG. 1, a variant thereof or a functional equivalent thereof.

3. A protein, a variant thereof or a functional equivalent thereof according to claim 1 or claim 2 having a molecular weight of about 139 kDa.

4. A tyrosine kinase modulator protein comprising the amino acid sequence given in FIG. 2, a variant thereof or a functional equivalent thereof.

5. A tyrosine kinase modulator protein consisting of the amino acid sequence given in FIG. 2, a variant thereof or a functional equivalent thereof.

6. A protein, a variant thereof or a functional equivalent thereof according to claim 5 having a molecular weight of about 56 kDa.

7. A protein, a variant thereof or a functional equivalent thereof according to any one of claims 1-6 that forms multimers.

8. A protein, a variant thereof or a functional equivalent thereof according to any one of claims 1-7 wherein said protein binds to an SH3 domain of a tyrosine kinase.

9. A protein, a variant thereof or a functional equivalent thereof according to any one of claims 1-8 wherein said tyrosine kinase is Abl, Src or Fyn.

10. A protein, a variant thereof or a functional equivalent thereof according to any one of claims 1-7 which binds the catalytic domain of Abl.

11. A protein, a variant thereof or a functional equivalent thereof according to any one of claims 1-10 which is a recombinant protein.

12. A protein, a variant thereof or a functional equivalent thereof according to any one of claims 1-10 which is genetically or chemically fused to one or more peptides or polypeptides.

13. A protein, a variant thereof or a functional equivalent thereof according to claim 12 which is fused to a GST.

14. A nucleic acid molecule encoding a protein, a variant thereof or a functional equivalent thereof according to any one of claims 1-13.

15. A vector comprising a nucleic acid molecule according to claim 14.

16. A host cell transformed or transfected with a vector of claim 15.

17. A transgenic animal that has been transformed by a nucleic acid molecule according to claim 16.

18. A method of preparing a tyrosine kinase modulator protein, a variant thereof or a functional equivalent thereof comprising expressing a vector according to claim 15 in a host cell, culturing said host cell under conditions wherein said tyrosine kinase modulator protein, variant thereof or functional equivalent thereof is expressed, and recovering said modulator protein, variant thereof or functional equivalent thereof.

19. A ligand which binds to a protein, a variant thereof or a functional equivalent thereof according to any one of claims 1-13, wherein said ligand is not a tyrosine kinase.

20. A ligand according to claim 19, which is an antibody.

21. A ligand according to either claim 19 or claim 20, which is fused to a label.

22. A ligand according to claim 21 wherein said label is radioactive, fluorescent, enzymatic, a toxin or an antibody.

23. A ligand according to any one of claims 19-22 for use in modulating tyrosine kinase activity.

24. A pharmaceutical composition comprising a protein, a variant thereof or a functional equivalent thereof according to any one of claims 1-13, a nucleic acid molecule according to claim 14, a vector according to claim 15 or a ligand according to any one of claims 19-23 in conjunction with a pharmaceutically acceptable carrier.

25. A protein, a variant thereof or a functional equivalent thereof according to any one of claims 1-13, a nucleic acid molecule according to claim 14, a vector according to claim 15, a ligand according to any one of claims 19-23 or a pharmaceutical composition according to claim 24 for use in the treatment or diagnosis of disease.

26. A method of treating disease comprising administering to a subject an effective dose of a protein, a variant thereof or a functional equivalent thereof according to any one of claims 1-13, a nucleic acid molecule according to claim 14, a vector according to claim 15, a ligand according to any one of claims 19-25 or a pharmaceutical composition according to claim 26.

27. Use of a protein, a variant thereof or a functional equivalent thereof according to claims 1-13, a nucleic acid molecule according to claim 14, a vector according to claim 15, a ligand according to any one of claims 19-25 or a pharmaceutical composition according to claim 26 in the manufacture of a medicament for treating or diagnosing cancer.

28. A method or use according to either claim 26 or claim 27, wherein said cancer is leukaemia.

29. A process for the formulation of a composition according to claim 24, comprising bringing a protein, variant thereof or functional equivalent thereof according to claims 1-13, a nucleic acid molecule according to claim 14, a vector according to claim 15 or a ligand according to any one of claims 19-23 into association with a pharmaceutically acceptable carrier.

30. A method for screening for a small molecule drug ligand capable of interaction with a protein, variant or functional equivalent according to any one of claims 1-13, said method comprising contacting a candidate ligand with said protein, variant or functional equivalent and selecting a ligand that demonstrates a binding response, or that stimulates or inhibits a functional response, compared to a control where the protein, variant or functional equivalent is not contacted with the candidate ligand.
